(19) United States
(12) Patent Application Publication
(10) Pub. No.: US 2022/0331150 A1
(43) Pub. Date: Oct. 20, 2022

(54) EMBEDDED PROTRUSION INTEGRATED CONDOM, AND MANUFACTURING PROCESS AND SPECIAL PRODUCTION EQUIPMENT THEREFOR

(71) Applicant: WENLONG GAO, Guangzhou (CN)

(72) Inventor: WENLONG GAO, Guangzhou (CN)

(21) Appl. No.: 17/378,766

(22) Filed: Jul. 19, 2021

(30) Foreign Application Priority Data
Apr. 16, 2021 (CN) 202110408513.6

(51) Int. Cl. A61F 6/04; B29D 23/00

(52) U.S. Cl. CPC A61F 6/04; B29D 23/00; A61F 2006/048; B29K 2007/00

(57) ABSTRACT

An embedded protrusion integrated condom, a manufacturing process, and a piece of special production equipment are disclosed. The condom includes a condom body and a function adjusting component configured to adjust the usage effect. The manufacturing process includes a condom body forming step, a function part paste step, a function part embedding step, and an embedded condom body structure reinforcement step. The special production equipment includes a multi-station synchronous impregnation mold, at least one series of combined impregnation pool, a control system, an automatic latex brush mechanism matching the combined impregnation pool, an automatic paste mechanism, and an automatic blanking mechanism. The condom has the usage effects such as good body touch effect and being able to realize physical delay; the manufacturing process and the special production equipment has the technical advantages of high automation degree, high production efficiency, and the like.

### CROSS-REFERENCE TO RELATED APPLICATIONS

This is a U.S. patent application which claims the priority and benefit of Chinese Patent Application Number 202110408513.6, filed on Apr. 16, 2021, the disclosure of which is incorporated herein by reference in its entirety.

### TECHNICAL FIELD

The present disclosure relates to the technical field of condoms (rain coats or rubbers, hereinafter referred to as condoms), and specifically relates to an embedded protrusion integrated condom, and a manufacturing process and special production equipment therefor.

### BACKGROUND

Existing ordinary condoms can only meet the needs of safe contraception, but women are often at a disadvantage in terms of sexual satisfaction in sexual life. One sex process ends at the end of male's ejaculation. Whether women can get stronger and longer-lasting stimulation to achieve harmony in their sexual life has become a pain point for people's needs. Therefore, the research and development of condoms will increase the friction of protrusions on the surface to enhance the stimulation.

For existing condoms provided with textures on the surfaces, such as particles and threads, concave and convex textures are made on a mold, and tiny textures are formed on the surfaces after impregnation. Due to the problems of adhesion of latex and flexibility after molding, as well as the mold processing difficulty and technical implementation difficulty, the existing ordinary condoms such as particles and threads have extremely fine surface protrusions, which basically do not exceed 2 mm.

Existing condoms that use glue to stick attachments in the later stage are peeled off from a production line after they are shaped, and then reattached to a fixed mold. The attachments are manually glued and then attached to the condoms. Uneven fitting, unstable sticking, or easy detachment from the condoms during use are easy to occur. Furthermore, the manufacturing process is complicated, and it is difficult to achieve efficient mass production.

For existing condoms with delay function, anesthetic drugs are added in the condoms mainly to paralyze male's penis and reduce the sensitivity to prolong the time of sex. However, the long-term use of the anesthetic drugs is burdensome, and a delay condom with the anesthetic drugs needs to wait for a certain period of time to take effect after being worn on male, and the experience effect is unstable. Moreover, the delay condoms with the anesthetic drugs are expensive.

### SUMMARY

In view of this, the present disclosure provides an embedded protrusion integrated condom, and a manufacturing process and special production equipment therefor, so as to solve the technical problems in the existing art.

The present disclosure provides an embedded protrusion integrated condom provided to solve the technical problems is that:

an embedded protrusion integrated condom includes a condom body and a function adjusting component used to adjust the usage effect. The condom body and the function adjusting component are integrated.

In one embodiment, the function adjusting component is flexible rubber sheet and/or a flexible rubber ring.

In one embodiment, the flexible rubber sheet includes a rubber sheet base film and a plurality of flexible rubber protrusions, and the flexible rubber protrusions are integrated on the rubber sheet base film.

In one embodiment, the height of each flexible rubber protrusion is not less than 2 mm.

In one embodiment, the flexible rubber ring includes a circular ring body; and the circular ring body has a length of not less than 20 mm and a thickness of not less than 1.5 mm.

In one embodiment, the condom body includes a base film layer, an embedded film layer and a reinforcement film layer; the base film layer, the embedded film layer and the reinforcement film layer are fused into a whole through an impregnation process; the function adjusting component is arranged between the base film layer and the embedded film layer, and is integrated with the base film layer, the embedded film layer and the reinforcement film layer through the impregnation process.

The present disclosure provides a manufacturing process for an embedded protrusion integrated condom provided to solve the technical problems is that:

a manufacturing process for an embedded protrusion integrated condom includes a condom body forming step, a function part paste step, a function part embedding step, and an embedded condom body structure reinforcement step. At the condom body forming step, a condom forming mold is used to perform condom body forming impregnation operation to form a condom body; at the function part paste step, a function adjusting component is pasted to the condom body formed at the condom body forming step; at the function part embedding step, embedding impregnation operation is performed on the condom body with the function adjusting component to form an embedded condom body structure; and at the embedded condom body structure reinforcement step, reinforcement impregnation operation is performed on the condom body subjected to the embedding impregnation operation to integrate the function adjusting component and the condom body.

In one embodiment, a conjunctival type latex or emulsion formula is used in the condom body forming impregnation operation, the embedding impregnation operation and the reinforcement impregnation operation; and the conjunctival type latex or emulsion formula includes a condom body main ingredient, a vulcanizing agent, a stabilizer, a dispersing agent, and an anti-aging agent.

In one embodiment, the condom body main ingredient is one of or any combination of natural latex, a polyurethane (PU) material and a polyisoprene (PI) material.

In one embodiment, the function part paste step further includes a location latex brush step used to brush latex to the condom body before the function adjusting component is pasted.

The present disclosure provides special production equipment for an embedded protrusion integrated condom provided to solve the technical problems is that:

special production equipment for an embedded protrusion integrated condom includes a multi-station synchronous impregnation mold, at least one series of combined impregnation pool, a control system, an automatic latex brush mechanism matching the combined impregnation pool, an automatic paste mechanism, and an automatic blanking mechanism. The multi-station synchronous impregnation mold is used to cooperate with the combined impregnation pool to synchronously complete the condom body forming operation, the embedded structure forming operation and the condom body structure reinforcement forming operation of at least two stations; each series of combined impregnation pool includes a condom body forming impregnation pool, an embedding impregnation pool and a reinforcement impregnation pool; the condom body forming impregnation pool, the embedding impregnation pool and the reinforcement impregnation pool are respectively used to complete the condom body forming operation, the embedding impregnation and the condom body structure reinforcement forming operation; the automatic latex brush mechanism is used to complete the automatic latex brush operation of the condom body of the embedded protrusion integrated condom to realize pasting of embedded protrusions; the automatic paste mechanism is used to complete automatic loading and automatic pasting of the embedded protrusions; the automatic blanking mechanism is used to complete automatic blanking of the embedded protrusion integrated condom finished product; and the control system is used to provide a system control function to the special production equipment for the embedded protrusion integrated condom.

In one embodiment, the multi-station synchronous impregnation mold includes a multi-station synchronization seat, a mold fixing seat, a mold lifting frame, a lifting driving mechanism, and a mold mounting mechanism; the mold mounting mechanism is arranged on the mold lifting frame; the mold lifting frame is slidably connected to the mold fixing seat; the mold lifting frame can rise up or drop down along the mold fixing seat under the action of the lifting driving mechanism to drive the mold mounting mechanism to do synchronous lifting motion; the mold fixing seat is arranged on the multi-station synchronization seat, and can do rotating motion under the action of the multi-station synchronization seat to drive the mold lifting frame to do synchronous rotating motion.

In one embodiment, the automatic latex brush mechanism includes an automatic latex brush, a latex brush telescopic arm and a telescopic arm driving pressure cylinder; the automatic latex brush is arranged at one end of the latex brush telescopic arm; the other end of the latex brush telescopic arm is fixed on the telescopic arm driving pressure cylinder; the automatic latex brush can extend or retract under the action of the telescopic arm driving pressure cylinder to complete brushing and resetting.

In one embodiment, the automatic paste mechanism includes a loading push arm, a loading template, a tumbler mechanism, a push arm driving pressure cylinder, an automatic storage disk, a transfer robot arm and a material suction cup; the loading template is arranged at one end of the loading push arm, and the other end of the loading push arm is arranged on the tumbler mechanism; the tumbler mechanism is arranged on the push arm driving pressure cylinder; the loading push arm can do linear motion of moving forward or backward under the action of the push arm driving pressure cylinder, and can also do rotating motion under the action of the tumbler mechanism; and the loading template can do synchronous linear motion or rotating motion under the action of the loading push arm. The automatic storage disk is used to store and stack a material, i.e., a function adjusting component; the transfer robot arm is used to realize transferring of the material from the automatic storage disk to the loading template; the material suction cup is arranged on the transfer robot arm and used to suck the material in the automatic storage disk.

In one embodiment, the automatic blanking mechanism includes a first blanking plate, a second blanking plate, a first blanking plate driving mechanism, a second blanking plate driving mechanism, a chute and a finished product bin; the first blanking plate and the second blanking plate are respectively arranged above the reinforcement impregnation pool; the first blanking plate driving mechanism and the second blanking plate driving mechanism are respectively used to drive the first blanking plate and the second blanking plate to move towards each other in opposite directions or do relative motion; the first blanking plate and the second blanking plate can form a blanking position when being closed; and the first blanking plate and the second blanking plate can give way to the condom body structure reinforcement forming operation when being separated.

Beneficial Technical Effects:

1. According to the embedded protrusion integrated condom and the manufacturing process therefor which are provided by the present disclosure, the pre-manufactured protrusions (rubber blocks, rubber sheets, rubber rings, etc.) are embedded into any interval positions (head, middle, bottom) of the condom or any surface position of the condom cylinder, so that the condom has a better body touch effect and can obviously reduce the male's sensitivity to realize a physical delay function, thereby effectively improving the usage effect of the condom and improving the quality of sex life.

2. The special production equipment for the embedded protrusion integrated condom provided by the present disclosure can realize high-efficiency and high-quality production of the embedded protrusion integrated condom by means of the cooperation of the multi-station synchronous impregnation mold, the combined impregnation pools, the automatic latex brush mechanism, the automatic paste mechanism, the automatic blanking mechanism, and the control system, and has the technical advantages of high automation degree, high production efficiency and easy realizing of batch production, and the like.

The technical solutions and technical effects of the present disclosure are described in detail below in combination with the specification drawings and specific implementation modes.

### BRIEF DESCRIPTION OF THE DRAWINGS

FIG. 1 illustrates a technological process and a schematic structural diagram of a product according to a first illustrative embodiment.

FIG. 2 illustrates a technological process and a schematic structural diagram of a product according to a second illustrative embodiment.

FIG. 3 illustrates a technological process and a schematic structural diagram of a product according to a third illustrative embodiment.

FIG. 4 illustrates a flowchart of a manufacturing process of an embedded protrusion integrated condom.

FIG. 5 illustrates a two-dimensional schematic diagram of special production equipment for an embedded protrusion integrated condom.

FIG. 6 illustrates a schematic diagram of a side surface of special production equipment for an embedded protrusion integrated condom.

FIG. 7 illustrates a partially enlarged diagram of part A shown in FIG. 5.

10: condom body; 20: function adjusting component; 110: base film layer; 120: embedded film layer; 130: reinforcement film layer; 210: flexible rubber sheet; 220: flexible rubber ring; 2110: rubber sheet base film; 2120: flexible rubber protrusion; 1: multi-station synchronous impregnation mold; 2: combined impregnation pool; 3: automatic paste mechanism; 4: automatic blanking mechanism; 11: multi-station synchronization seat; 12: mold fixing seat; 13: mold lifting frame; 14: lifting driving mechanism; 15: mold mounting mechanism; 1410: lifting driving motor; 1420: lifting driving screw rod; 1430: lifting driving sliding table; 21: condom body forming impregnation pool; 22: embedding impregnation pool; 23: reinforcement impregnation pool; 31: loading push arm; 32: loading template; 33: tumbler mechanism; 34: push arm driving pressure cylinder; 35: automatic storage disk; 36: transfer robot arm; 37: material suction cup; 321: flexible rubber sheet placement position; 322: flexible rubber ring placement position; 323: condom body through hole; 41: first blanking plate; 42: second blanking plate; 43: first blanking plate driving mechanism; 44: second blanking plate driving mechanism; 45: chute; 46: finished product bin.

### DESCRIPTION OF THE EMBODIMENTS

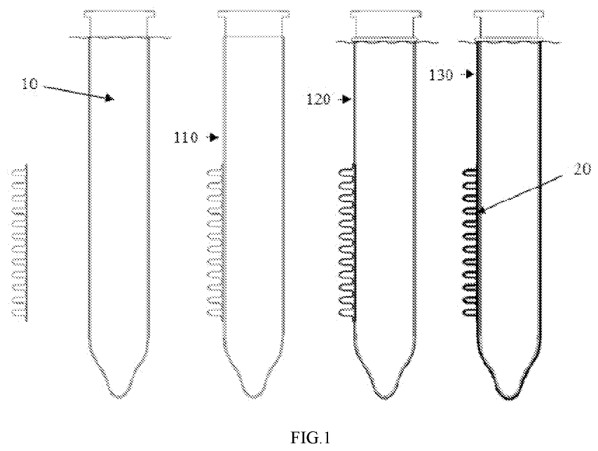

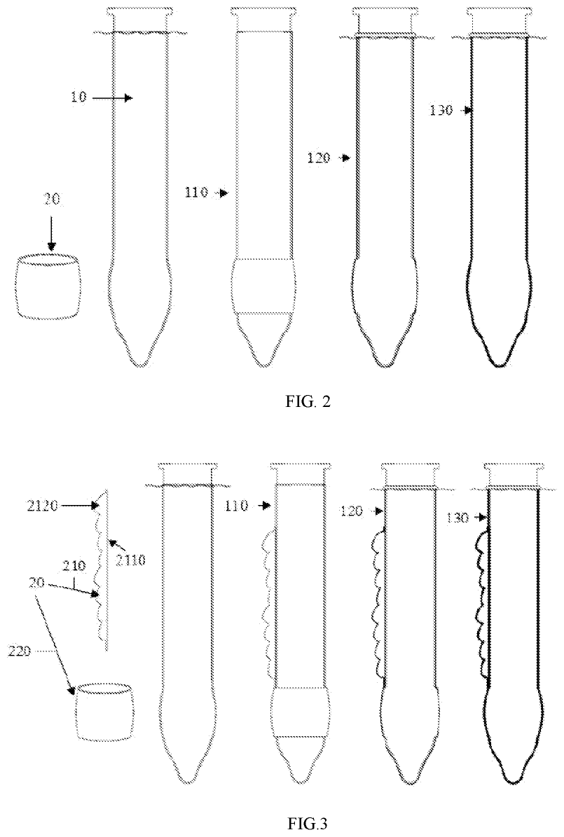

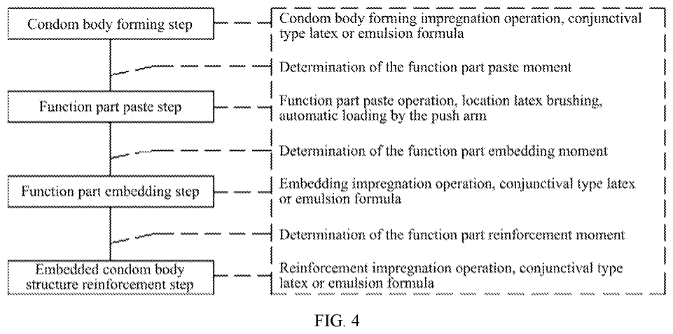

Referring to FIG. 1 through FIG. 3, an embedded protrusion integrated condom provided by the present disclosure includes a condom body 10 and a function adjusting component 20 used to adjust the usage effect. The condom body 10 and the function adjusting component 20 are integrated. The function adjusting component 20 is a flexible rubber sheet 210 (FIG. 1), or a flexible rubber ring 220 (FIG. 2) or includes both the flexible rubber sheet 210 and the flexible rubber ring 220 (FIG. 3). The flexible rubber sheet 210 includes a rubber sheet base film 2110 and a plurality of flexible rubber protrusions 2120, and the flexible rubber protrusions 2120 are integrated on the rubber sheet base film 2110. The height of each flexible rubber protrusion 2120 is not less than 2 mm. The flexible rubber ring 220 includes a circular ring body; and the circular ring body has a length of not less than 20 mm and a thickness of not less than 1.5 mm.

The condom body 10 includes a base film layer 110, an embedded film layer 120 and a reinforcement film layer 130. The base film layer 110, the embedded film layer 120 and the reinforcement film layer 130 are fused into a whole through an impregnation process; the function adjusting component 20 is arranged between the base film layer 110 and the embedded film layer 120, and is integrated with the base film layer 110, the embedded film layer 120 and the reinforcement film layer 130 through the impregnation process.

The embedded protrusion integrated condom of the present disclosure is manufactured by the following process: a condom body forming step, where a condom forming mold is used to perform condom body forming impregnation operation to form the condom body 10 and the base film layer 110 of the condom body 10;

a function part paste step, where the function adjusting component 20 is pasted to the condom body 10 formed at the condom body forming step, and the function adjusting component can be the flexible rubber sheet 210 and/or the flexible rubber ring 220;

a function part embedding step, where embedding impregnation operation is performed on the condom body 10 with the function adjusting component 20 to form an embedded condom body structure, and the embedded film layer 120 is formed on the base film layer 110 and the function adjusting component 20; and

an embedded condom body structure reinforcement step, where reinforcement impregnation operation is performed on the condom body 10 subjected to the embedding impregnation operation to integrate the function adjusting component 20 and the condom body 10, and the reinforcement film layer 130 is formed on the embedded film layer 120.

A conjunctival type latex or emulsion formula is used in the condom body forming impregnation operation, the embedding impregnation operation and the reinforcement impregnation operation; and the conjunctival type latex or emulsion formula includes a condom body main ingredient, a vulcanizing agent, a stabilizer, a dispersing agent, and an anti-aging agent. The condom body main ingredient is one of or any combination of natural latex, a polyurethane (PU) material and a polyisoprene (PI) material.

In some improved embodiments, the function part paste step further includes a location latex brush step used to brush latex to the condom body 10 before the function adjusting component 20 is pasted to improve the function part paste effect.

Explanation of the technical effects: according to the embedded protrusion integrated condom and the manufacturing process therefor which are provided by the present disclosure, the pre-manufactured protrusions (rubber blocks, rubber sheets, rubber rings, etc.) are embedded into any interval positions (head, middle, bottom) of the condom or any surface position of the condom cylinder, so that the condom has a better body touch effect and can obviously reduce the male's sensitivity to realize a physical delay function, thereby effectively improving the usage effect of the condom and improving the quality of sex life.

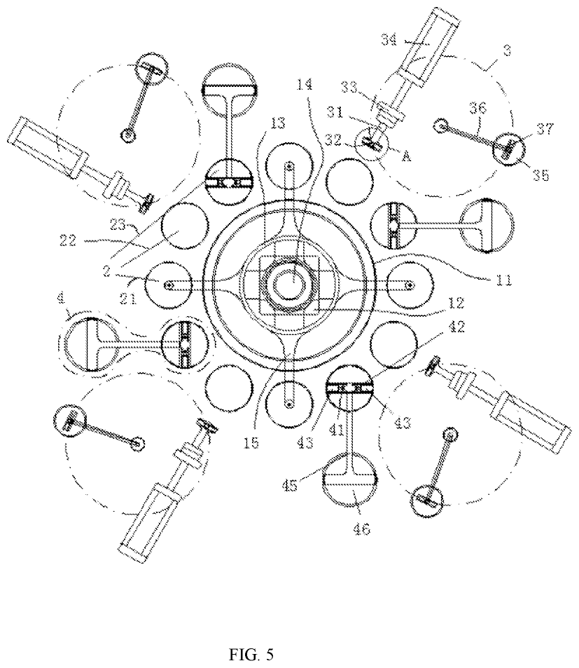

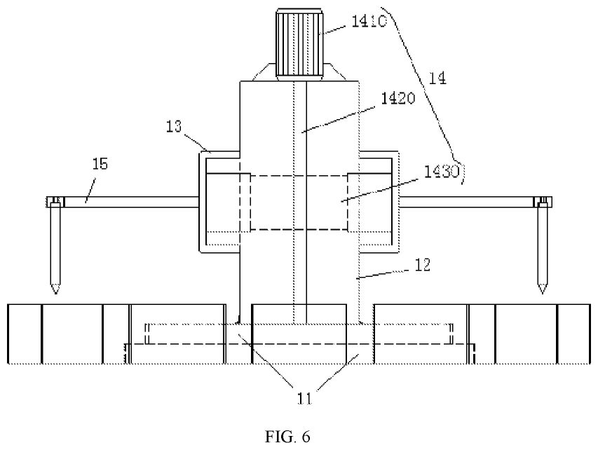

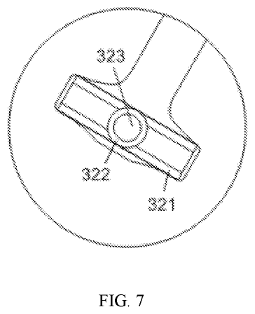

Referring to FIG. 5 to FIG. 6, consistent with the foregoing condom manufacturing process, the present disclosure further discloses special production equipment for an embedded protrusion integrated condom. The equipment includes a multi-station synchronous impregnation mold 1, at least one series of combined impregnation pool 2, a control system, an automatic latex brush mechanism matching the combined impregnation pool 2, an automatic paste mechanism 3, and an automatic blanking mechanism 4.

The multi-station synchronous impregnation mold 1 includes a four-station synchronous impregnation mold; there are four series of combined impregnation pools 2; each combined impregnation pool 2 includes a condom body forming impregnation pool 21, an embedding impregnation pool 22 and a reinforcement impregnation pool 23. Each embedding impregnation pool 22 is provided with an automatic paste mechanism 3, and each reinforcement impregnation pool 23 is provided with an automatic blanking mechanism 4.

The multi-station synchronous impregnation mold 1 is used to cooperate with the combined impregnation pools 2 to synchronously complete the condom body forming operation, the embedded structure forming operation and the condom body structure reinforcement forming operation of four stations. The multi-station synchronous impregnation mold 1 includes a multi-station synchronization seat 11, a mold fixing seat 12, a mold lifting frame 13, a lifting driving mechanism 14, and a mold mounting mechanism 15; the mold mounting mechanism 15 is arranged on the mold lifting frame 13; the mold lifting frame 13 is slidably connected to the mold fixing seat 12; the mold lifting frame 13 can rise up or drop down along the mold fixing seat 12 under the action of the lifting driving mechanism 14 to drive the mold mounting mechanism 15 to do synchronous lifting motion; the mold fixing seat 12 is arranged on the multi-station synchronization seat 11, and can do rotating motion under the action of the multi-station synchronization seat 11 to drive the mold lifting frame 13 to do synchronous rotating motion. Specifically, the multi-station synchronization seat 11 can be various index plates such as an electric index plate, and generally includes a fixed seat, a rotating seat and a rotating driving motor, where the rotating seat can do rotating motion at any angle under the driving of the rotating driving motor.

The condom body forming impregnation pool 21, the embedding impregnation poor 22 and the reinforcement impregnation pool 23 of each series are respectively used to complete the condom body forming operation, the embedding impregnation operation and the condom body structure reinforcement forming operation of this series.

The automatic latex brush mechanism (not shown in the figure) is used to complete the automatic latex brush operation of the condom body of the embedded protrusion integrated condom to realize pasting of the embedded protrusions; and a combined mechanism of the brush, the telescopic arm and a pressure cylinder such as an air cylinder and a hydraulic cylinder can be used to complete the automatic latex brush operation. For example, the brush is arranged at one end of the telescopic arm, and the other end of the telescopic arm is fixed on the pressure cylinder; and the brush can extend or retract under the action of the pressure cylinder to complete latex brushing and resetting.

The automatic paste mechanism 3 is used to complete automatic loading and automatic pasting of the embedded protrusions. The automatic paste mechanism 3 includes a loading push arm 31, a loading template 32, a tumbler mechanism 33, a push arm driving pressure cylinder 34, an automatic storage disk 35, a transfer robot arm 36, and a material suction cup 37; the loading template 32 is arranged at one end of the loading push arm 31, and the other end of the loading push arm 31 is arranged on the tumbler mechanism 33; the tumbler mechanism 33 is arranged on the push arm driving pressure cylinder 34; the loading push arm 31 can do linear motion of moving forward or backward under the action of the push arm driving pressure cylinder 34, and can also do rotating motion under the action of the tumbler mechanism 33; and the loading template 32 can do synchronous linear motion or rotating motion under the action of the loading push arm 31. The loading template 32 can do synchronous linear motion or rotating motion under the action of the loading push arm 31; the automatic storage disk 35 is used to store and stack a material; the transfer robot arm 36 is used to realize transferring of the material from the automatic storage disk 35 to the loading template 32; and the material suction cup 37 is arranged on the transfer robot arm 36 and used to suck the material in the automatic storage disk 35. Specifically, the tumbler mechanism 33 can be various index plate such as an electric index plate; the push arm driving pressure cylinder 34 can be various air cylinders or hydraulic cylinders; the automatic storage disk 35 can be various vibration feeding disks, vibration loading disks or vibration loading machines; the transfer robot arm 36 can be various rotating mechanical arms or robots; and the material suction cup 37 can be various vacuum suction cups or vacuum suction nozzles that use a vacuum suction force to realize material suction. The automatic paste mechanism 3 is arranged around the embedding impregnation pool 22. Explanation of an automatic paste principle: a long-strip type flexible rubber sheet placement position 321 is arranged on the loading template 32; the flexible rubber sheet placement position 321 is provided with a flexible rubber ring placement position 322 in the middle; and the flexible rubber ring placement position 322 is provided with a condom body through hole 323 in the middle. When the function adjustment part to be pasted is a flexible rubber sheet, the material suction cup 37 places the flexible rubber sheet to the flexible rubber sheet placement position 321; the loading push arm 31 pushes the loading template 32 to be around the condom body; and the loading template 32 is flipped to the condom body to paste the flexible rubber sheet to the condom body. When the function adjustment part to be pasted is a flexible rubber ring 32, the material suction cup 37 places the flexible rubber ring to the flexible rubber ring placement position 322; the condom body rises up under the driving of the condom mold; the loading push arm 31 pushes the loading template 32 to be right under the condom body; the condom body then drops down and passes through the condom body through hole 323; and the flexible rubber ring is pasted on the condom body.

The automatic blanking mechanism 4 is used to complete automatic blanking of the embedded protrusion integrated condom finished product. The automatic blanking mechanism 4 includes a first blanking plate 41, a second blanking plate 42, a first blanking plate driving mechanism 43, a second blanking plate driving mechanism 44, a chute 45 and a finished product bin 46; the first blanking plate 41 and the second blanking plate 42 are respectively arranged above the reinforcement impregnation pool 23; the first blanking plate driving mechanism 43 and the second blanking plate driving mechanism 44 are respectively used to drive the first blanking plate 41 and the second blanking plate 42 to move towards each other in opposite directions or do relative motion; the first blanking plate 41 and the second blanking plate 42 can form a blanking position and a chute inlet structure when being closed; and the first blanking plate 41 and the second blanking plate 42 can give way to the condom body structure reinforcement forming operation when being separated. Specifically, cooperation parts of the first blanking plate 41 and the second blanking plate 42 are of semicircular structures and are both flexible rubber; and the first blanking plate driving mechanism 43 and the second blanking plate driving mechanism 44 are both air cylinders or hydraulic cylinders. The first blanking plate driving mechanism 43 and the second blanking plate driving mechanism 44 are arranged at the upper part of the reinforcement impregnation pool; and the first blanking plate 41 and the second blanking plate 42 are respectively arranged on piston rods or telescopic rods of the first blanking plate driving mechanism 43 and the second blanking plate driving mechanism 44. Explanation of an automatic blanking principle: during the reinforcement impregnation operation, the first blanking plate 41 and the second blanking plate 42 are separated from each other to give way to the reinforcement impregnation operation of the condom mold; after the reinforcement impregnation operation is completed and the condom body is cured and sized, the first blanking plate 41 and the second blanking plate 42 are closed to form the blanking position and the chute inlet structure; the condom mold rises up and passes through the blanking position to remove the condom from the mold; and the condom separated from the mold passes through the chute 45 and enters the finished product bin 46.

Driving components of the multi-station synchronization seat 11, such as a control port of the rotating driving motor, a control port of the telescopic arm driving pressure cylinder of the automatic latex brush mechanism, control ports of the tumbler mechanism 33, the push arm driving pressure cylinder 34, the transfer robot arm 36 and the material suction cup 37 of the automatic paste mechanism 3, and control ports of the first blanking plate driving mechanism 43 and the second blanking plate driving mechanism 44 of the automatic blanking mechanism 4 are all electrically connected to the control system.

Explanation of an Equipment Operation Mechanism:

After the various materials are prepared and the various station molds are installed and debugged properly, the equipment is initiated;

the multi-station synchronous impregnation mold 1 completes the condom body forming impregnation operation of four stations through the rotating motion of the multi-station synchronization seat 11 and the lifting motion of the mold lifting frame 13 to form the base film 110 of the condom body 10 of the condom, which corresponds to the condom body forming step;

the automatic latex brush mechanism of each station completes the automatic latex brush operation of the base film 110 of the condom body; the automatic paste mechanism 6 completes automatic loading and paste operations of the function adjusting component through coordinating cooperation of the loading push arm 31, the loading template 32, the tumbler mechanism 33, the push arm driving pressure cylinder 34, the automatic storage disk 35, the transfer robot arm 36 and the material suction cup 37, which corresponds to the function part paste step;

the control system instructs the multi-station synchronization seat 11 of the multi-station synchronous impregnation mold 1 rotates according to a specified angle to transfer the condom body forming mold of each station to be above the embedding impregnation pool 22 of each station and then completes the embedding impregnation operation through the lifting motion of the mold lifting frame 13; the embedded film layer 120, i.e., an embedded condom body structure, is formed on the base film layer 110 of the condom body and the function adjusting component 20, which corresponds to the function part embedding step;

the control system instructs the multi-station synchronization seat 11 of the multi-station synchronous impregnation mold 1 rotates according to a specified angle to transfer the condom body forming mold of each station to be above the reinforcement impregnation pool 23 of each station and then completes the reinforcement impregnation operation through the lifting motion of the mold lifting frame 13; the reinforcement film layer 130, i.e., an embedded condom body structure, is formed on the embedded film layer 120 of the condom body, which corresponds to the embedded condom body structure reinforcement step;

after the reinforcement impregnation operation is completed and the condom body is solidified and sized, the automatic blanking mechanism 4 forms the blanking position and the chute inlet structure through the cooperation of the first blanking plate 41 and the second blanking plate 42 and the cooperation of the first blanking plate driving mechanism 43 and the second blanking plate driving mechanism 44, so as to automatically realize hemming, demolding and warehousing of finished products.

Explanation of technical effects: The special production equipment for the embedded protrusion integrated condom provided by the present disclosure can realize high-efficiency and high-quality production of the embedded protrusion integrated condom of the present disclosure by means of the cooperation of the multi-station synchronous impregnation mold, the combined impregnation pools, the automatic latex brush mechanism, the automatic paste mechanism, the automatic blanking mechanism, and the control system, and has the technical advantages of high automation degree, high production efficiency and easy realizing of batch production, and the like.

In some improved embodiments, the special production equipment for the embedded protrusion integrated condom provided by the present disclosure further includes a vision analysis mechanism and a vision analysis module. The vision analysis mechanism uses a plurality of industrial cameras to collect vision information of the condom body forming operation, the embedding impregnation operation and the condom body structure reinforcement forming operation; and the vision analysis module is arranged in the control system and determines an impregnation operation moment through vision information analysis.

The technical solutions and technical effects of the present disclosure are described in detail above in conjunction with the drawings and specific embodiments of the specification. It should be noted that the specific implementation modes disclosed in the specification are only illustrative embodiments of the present disclosure. Those skilled in the art can also develop other embodiments on this basis; any simple modifications and equivalent replacements that do not depart from the innovative concept of the present disclosure are covered by the present disclosure and belong to the protection scope of this patent.

What is claimed is:

1. An embedded protrusion integrated condom, comprising a condom body (10) and a function adjusting component (20) configured to adjust a usage effect, wherein the condom body (10) and the function adjusting component (20) are integrated with each other.

2. The embedded protrusion integrated condom of claim 1, wherein the function adjusting component (20) comprises at least one selected from the group consisting of a flexible rubber sheet (210) and a flexible rubber ring (220).

3. The embedded protrusion integrated condom of claim 2, wherein the flexible rubber sheet (210) comprises a rubber sheet base film (2110) and a plurality of flexible rubber protrusions (2120), and wherein the flexible rubber protrusions (2120) are integrated on the rubber sheet base film (2110).

4. The embedded protrusion integrated condom of claim 3, wherein each flexible rubber protrusion (2120) has a height of not less than 2 mm.

5. The embedded protrusion integrated condom of claim 2, wherein the flexible rubber ring (220) comprises a circular ring body, which has a length of not less than 20 mm and a thickness of not less than 1.5 mm.

6. The embedded protrusion integrated condom of claim 1, wherein the condom body (10) comprises a base film layer (110), an embedded film layer (120) and a reinforcement film layer (130);
wherein the base film layer (110), the embedded film layer (120) and the reinforcement film layer (130) are fused into a whole through an impregnation process; the function adjusting component (20) is arranged between the base film layer (110) and the embedded film layer (120), and is integrated with the base film layer (110), the embedded film layer (120) and the reinforcement film layer (130) through the impregnation process.

7. A manufacturing process for an embedded protrusion integrated condom, comprising:
a condom body forming step, in which a condom forming mold is used to perform a condom body forming impregnation operation to form a condom body (10);
a function part paste step, in which a function adjusting component (20) is pasted to the condom body (10) formed at the condom body forming step;
a function part embedding step, in which an embedding impregnation operation is performed on the condom body (10) with the function adjusting component (20) to form an embedded condom body structure; and
an embedded condom body structure reinforcement step, in which a reinforcement impregnation operation is performed on the condom body (10) subjected to the embedding impregnation operation to integrate the function adjusting component (20) and the condom body (10).

8. The manufacturing process of claim 7, wherein a conjunctival type latex or emulsion formula is used in the condom body forming impregnation operation, the embedding impregnation operation and the reinforcement impregnation operation; and the conjunctival type latex or emulsion formula comprises a condom body main ingredient, a vulcanizing agent, a stabilizer, a dispersing agent, and an anti-aging agent.

9. The manufacturing process of claim 8, wherein a condom body main ingredient comprises at least one selected from the group consisting of a natural latex, a polyurethane (PU) material, and a polyisoprene (PI) material.

10. The manufacturing process of claim 7, wherein the function part paste step further comprises a location latex brush step used to brush latex to the condom body (10) before the function adjusting component (20) is pasted.

11. A piece of special production equipment for an embedded protrusion integrated condom, comprising a multi-station synchronous impregnation mold (1), at least one series of combined impregnation pool (2), a control system, an automatic latex brush mechanism matching the combined impregnation pool (2), an automatic paste mechanism (3), and an automatic blanking mechanism (4),
wherein the multi-station synchronous impregnation mold (1) is configured to cooperate with the combined impregnation pool (2) to synchronously perform the condom body forming operation, the embedded structure forming operation and the condom body structure reinforcement forming operation of at least two stations;
each series of combined impregnation pool (2) comprises a condom body forming impregnation pool (21), an embedding impregnation pool (22) and a reinforcement impregnation pool (23); the condom body forming impregnation pool (21), the embedding impregnation pool (22) and the reinforcement impregnation pool (23) are respectively configured to complete the condom body forming operation, the embedding impregnation and the condom body structure reinforcement forming operation;
the automatic latex brush mechanism is configured to perform the automatic latex brush operation of the condom body of the embedded protrusion integrated condom to achieve pasting of embedded protrusions;
the automatic paste mechanism (3) is configured to perform automatic loading and automatic pasting of the embedded protrusions;
the automatic blanking mechanism (4) is configured to perform automatic blanking of the embedded protrusion integrated condom finished product; and the control system is configured to provide a system control function to the special production equipment for the embedded protrusion integrated condom.

12. The special production equipment of claim 11, wherein the multi-station synchronous impregnation mold (1) comprises a multi-station synchronization seat (11), a mold fixing seat (12), a mold lifting frame (13), a lifting driving mechanism (14), and a mold mounting mechanism (15); the mold mounting mechanism (15) is arranged on the mold lifting frame (13); the mold lifting frame (13) is slidably connected to the mold fixing seat (12); the mold lifting frame (13) is operative to rise up or drop down along the mold fixing seat (12) under the action of the lifting driving mechanism (14) to drive the mold mounting mechanism (15) to do synchronous lifting motion; the mold fixing seat (12) is arranged on the multi-station synchronization seat (11), and may do rotating motion under the action of the multi-station synchronization seat (11) to drive the mold lifting frame (13) to perform a synchronous rotating motion;
wherein the automatic latex brush mechanism comprises an automatic latex brush, a latex brush telescopic arm and a telescopic arm driving pressure cylinder; the automatic latex brush is arranged at one end of the latex brush telescopic arm; the other end of the latex brush telescopic arm is fixed on the telescopic arm driving pressure cylinder; the automatic latex brush is operative to extend or retract under the action of the telescopic arm driving pressure cylinder to complete brushing and resetting; the automatic paste mechanism (3) comprises a loading push arm (31), a loading template (32), a tumbler mechanism (33), a push arm driving pressure cylinder (34), an automatic storage disk (35), a transfer robot arm (36) and a material suction cup (37); the loading template (32) is arranged at one end of the loading push arm (31), and the other end of the loading push arm (31) is arranged on the tumbler mechanism (33); the tumbler mechanism (33) is arranged on the push arm driving pressure cylinder (34); the loading push arm (31) is operative to perform a linear motion of moving forward or backward under the action of the push arm driving pressure cylinder (34), and is also operative to perform a rotating motion under the action of the tumbler mechanism (33); and the loading template (32) is operative to perform a synchronous linear motion or rotating motion under the action of the loading push arm (31); the automatic storage disk (35) is configured to store and stack a material; the transfer robot arm (36) is configured to achieve transferring of the material from the automatic storage disk (35) to the loading template (32); the material suction cup (37) is arranged on the transfer robot arm (36) and configured to suck up the material stored in the automatic storage disk (35);
the automatic blanking mechanism (4) comprises a first blanking plate (41), a second blanking plate (42), a first blanking plate driving mechanism (43), a second blanking plate driving mechanism (44), a chute (45) and a finished product bin (46); the first blanking plate (41) and the second blanking plate (42) are each arranged above the reinforcement impregnation pool (23); the first blanking plate driving mechanism (43) and the second blanking plate driving mechanism (44) are respectively configured to drive the first blanking plate (41) and the second blanking plate (42) to move towards each other in opposite directions or perform relative motion; the first blanking plate (41) and the second blanking plate (42) are operative to form a blanking position when being closed; and the first blanking plate (41) and the second blanking plate (42) are operative to give way to the condom body structure reinforcement forming operation when being separated.
